(19) United States
(12) Patent Application Publication
(10) Pub. No.: US 2014/0275829 A1
(43) Pub. Date: Sep. 18, 2014

(54) SLEEP STAGE ANNOTATION DEVICE

(71) Applicants: Igor Berezhnyy, Eindhoven (NL); Tim Elisabeth Joseph Weysen, Maastricht (NL)

(72) Inventors: Igor Berezhnyy, Eindhoven (NL); Tim Elisabeth Joseph Weysen, Maastricht (NL)

(73) Assignee: KONINKLIJKE PHILIPS N.V., EINDHOVEN (NL)

(21) Appl. No.: 14/116,449

(22) PCT Filed: May 8, 2012
(86) PCT No.: PCT/IB2012/052274

§ 371 Date: Nov. 8, 2013

(30) Foreign Application Priority Data
May 11, 2011 (EP) 11305571.9

(51) Int. Cl. A61B 5/00; A61B 5/053; A61B 7/04; A61B 5/01; A61B 5/11

(52) U.S. Cl. CPC A61B 5/6892; A61B 5/01; A61B 5/11; A61B 7/04; A61B 5/053; A61B 5/746; A61B 5/7405; A61B 5/4812
USPC 600/301; 600/393; 600/372

(57) ABSTRACT

The present invention is related to a sleep stage annotation system, said system having a plurality of sensors elements comprising differential electrodes, at least one sensor element comprising a ground electrode, transmitting means to transmit signals generated by the differential electrodes and the at least one ground electrode to a data recording unit, wherein) at least the sensor elements comprising the differential electrodes are arranged on a device capable of serving as a head or face support means, and methods using the same.

### FIELD OF THE INVENTION

The invention relates to the field of sleep stage annotation.

### BACKGROUND OF THE INVENTION

In clinical practice, sleep stage annotation (SSA) is typically performed by a certified expert on the basis of visual examination of electrophysiological signals. Traditionally, three primary measures have been used to define physiological sleep and the different physiological sleep stages. These are the electroencephalogram (“EEG”), which is a sum signal emanating largely from changes in voltage of the membranes of nerve cells, the electrooculogram (“EOG”), which records electrophysiological phenomena caused by eye movements, in which the eyeball acts like a small battery, with the retina negative relative to the cornea, in such way that an electrode placed on the skin near the eye will record a change in voltage as the eye rotates, and the electromyogram (“EMG”), which is a record of electrical activity emanating from active muscles, and can be recorded from electrodes on the skin surface overlying a muscle (typically recorded from a region under the chin).

In practice, the EEG, EOG, and EMG are simultaneously recorded so that relationships among the three can be seen immediately. In a state of wakefulness, the EEG alternates between two major patterns. One is low voltage (about 10-30 microvolts) fast (16-25 Hz (or cps; cycles per second) activity, often called an “activation” or a desynchronized pattern. The other is a sinusoidal 8-12 Hz pattern (most often 8 or 12 Hz) of about 20-40 microvolts which is called “alpha” activity. Typically, alpha activity is most abundant when the subject is relaxed and the eyes are closed. The activation pattern is most prominent when subjects are alert with their eyes open and they are scanning the visual environment.

In rapid eye movement (“REM”) sleep, the EEG reverts to a low voltage, mixed frequency pattern. Bursts of prominent rapid eye movements appear. The background EMG is virtually absent, but many small muscle twitches may occur against this low background.

REM sleep is classified into two categories: tonic and phasic. REM sleep in adult humans typically occupies 20-25% of total sleep, i.e., about 90-120 minutes of a night's sleep. During a normal night of sleep, humans usually experience about four or five periods of REM sleep; they are quite short at the beginning of the night and longer toward the end. During REM sleep, the activity of the brain's neurons is quite similar to that during waking hours; for this reason, the REM-sleep stage may be called paradoxical sleep. REM sleep is physiologically different from the other phases of sleep, which are collectively referred to as non-REM sleep (“NREM sleep”). Vividly recalled dreams mostly occur during REM sleep.

In stage 1 sleep (nomenclature according to [4]), alpha activity decreases, activation is scarce, and the EEG consists mostly of low voltage, mixed frequency activity, much of it at 3-7 Hz. REMs are absent, but slow rolling eye movements appear. The EMG signal is moderate to low compared to wakefulness (which is usually accompanied by a high tonic EMG).

In stage 2 sleep, bursts of distinctive 12-14 Hz sinusoidal waves called “sleep spindles” appear in the EEG against a continuing background of low voltage, mixed frequency activity. Eye movements are rare, and the EMG signal is low to moderate compared to wakefulness.

In stage 3 sleep, high amplitude (>75 mV), slow (0.5-2 Hz) waves called “delta waves” appear in the EEG; EOG and EMG continue as before.

In stage 4 sleep, there is a quantitative increase in delta waves so that they come to dominate the EEG tracing.

Under the AASM (American Academy of Sleep Medicine) standard of 2007, a similar nomenclature applies, under which stage N1 refers to the transition of the brain from alpha waves having a frequency of 8-13 Hz (common in the awake state) to theta waves having a frequency of 4-7 Hz. This stage is sometimes referred to as somnolence or drowsy sleep. Sudden twitches and hypnic jerks, also known as positive myoclonus, may be associated with the onset of sleep during N1. Some people may also experience hypnagogic hallucinations during this stage, which can be troublesome to them. During N1, the subject loses some muscle tone and most conscious awareness of the external environment.

Stage N2 is characterized by sleep spindles ranging from 11-16 Hz (most commonly 12-14 Hz) and K-complexes, i.e., conspicuous EEG waveforms which have been suggested to (i) suppress cortical arousal in response to stimuli that the sleeping brain evaluates, and (ii) aide sleep-based memory consolidation. During this stage, muscular activity as measured by EMG decreases, and conscious awareness of the external environment disappears. This stage occupies 45-55% of total sleep in adults.

Stage N3 (deep or slow-wave sleep) is characterized by the presence of a minimum of 20% delta waves ranging from 0.5-2 Hz and having a peak-to-peak amplitude >75 μV. (EEG standards define delta waves to be from 0-4 Hz, but sleep standards in both the original R&K, as well as the new 2007 AASM guidelines have a range of 0.5-2 Hz.) This is the stage in which parasomnias such as night terrors, nocturnal enuresis, sleepwalking, and somniloquy occur. The following table gives an overview of the different sleep stages and their classification according to the different nomenclatures:

TABLE 1

nomenclature

| | Rechtschaffen | AASM |
|---|---|---|
| stage | & Kales [4] | 2007 |
| wake | — | — |
| light sleep | S1/S2 | N1/N2 |
| deep sleep | S3/S4 | N3 |
| REM | | |

Automatic sleep stage annotation has emerged as a tool to assist sleep experts and to accelerate the analysis of EEG data. The advent of consumer products aimed at enhancing the sleep experience has fostered the need for home sleep monitoring solutions which can i) provide automatic SSA using sensors that minimally interfere with the sleep process, and ii) provide sleep stage information in real-time in order to be suitable for closed-loop sleep inducing solutions. SSA is, to date, a difficult and laborious process which is usually performed in sleeping laboratories. SSA is thus, in most cases, not available for consumer use.

One product currently available is marked as “Zeo Personal Sleep Coach” and distributed by Zeo, Inc. This device comprises a headband comprising three electrodes (two differential electrodes and one ground electrode) connected to a differential amplifier and a data logger. During sleep, such headband may slide off the head, which may lead to bad signals that cannot be evaluated. Further, such headband may affect sleep comfort. Another problem of such device is that the number of electrodes and the potential positions of the latter are highly restricted. This may affect signal quality because the system is not very flexible, as it does not provide any alternative electrodes in case one or more electrodes create poor signals.

### SUMMARY OF THE INVENTION

It is an object of the present invention to provide a sleep stage annotation system which overcomes disadvantages, or shortcomings, of devices known from the prior art. It is another object of the present invention to provide a sleep stage annotation system which is suitable for consumer use. It is yet another object of the present invention to provide a sleep stage annotation system which has good signal quality, high flexibility and high user comfort. These objects are achieved by a system and/or by a method according to the independent claims.

### BRIEF DESCRIPTION OF THE DRAWINGS

These and other aspects of the invention will be apparent from and elucidated with reference to the embodiments described hereinafter.

In the drawings:

FIG. 1 shows a sleep stage annotation system 10 according to the present invention, which adopts the form of a pillow 11, which is a preferred embodiment of the device capable of serving as a head or face support means. The system has a plurality of sensor elements 12 comprising differential electrodes, arranged in a grid-like manner. The system according to FIG. 1 has 48 sensor elements, out of which 32 comprise differential electrodes (16 EEG electrodes and 16 reference electrodes) and 16 comprise ground electrodes. Deviating from FIG. 1, the sleep stage annotation system according to the invention can comprise any technically conceivable number of sensor areas, which can be arranged in any technically conceivable manner. Further, the sensor areas can comprise other sensor types, like temperature sensors, pressure sensors, light sensors, microphones, and/or accelerometers, too.

FIG. 2 shows an EEG approach according to the present invention. Features and labels (from top-to bottom): (A) Signal power vs. frequency over time, (B) Low frequency (deeper sleep) power over time, (C) hypnogram plot. See further discussion below.

FIG. 3 shows a two-dimensional visualization of the feature vectors corresponding to each sleep stage that can be obtained using the technique t-Distributed Stochastic Neighbor Embedding technique. See further discussion below.

FIG. 4 shows power spectra of a C4-A1 measurement (FIG. 4A) and an EOGLeft-A1 measurement (FIG. 4B) and hypnograms, both groundtruth (provided by expert scorer) and estimated (FIG. 4C). See description of FIG. 7 for nomenclature “EOGLeft” relates to an electrode placed near the left eye, corresponding approximately, to Fp1 (see FIG. 7).

FIGS. 5A and 5B show a schematic illustration of one embodiment of the sleep stage annotation system according to the present invention. In this embodiment, the sensor elements are functionally arranged in grid 50 in fixed groups 51 comprising two differential electrodes (EEG, REF) and one ground electrode (GND) each. The functional correlation of the two differential electrodes and one ground electrode is fixed, i.e., signals from the respective electrodes are amplified by means of a differential amplifier 52 and the resulting signal is then recorded on one channel of a given data storage device. This requires a fixed wiring scheme of the respective electrodes and amplifiers. Said functional correlation coincides with a fixed spatial arrangement, in which the respective sensor elements 53 comprising the electrodes of each group are arranged, in vertical columns. In this embodiment, differential amplification can take place on-site, i.e., in the device capable of serving as a head or face support means.

In such an embodiment, the differential amplifiers 52 are integrated in said device capable of serving as a head or face support means for each group of electrodes, e.g., for each triplet (which means a group of three electrodes: EEG, REF and GND). Further, in this embodiment, differential amplification takes place in real-time, preferably. After recording, the data sets can be analyzed, and the recording which yields the best signal quality (S/N ratio, appearance of sleep-related signal patterns) can be selected for further analysis.

FIG. 6 shows a schematic illustration of another embodiment of the sleep stage annotation system according to the present invention. In this embodiment, the sensor elements are arranged in a grid 60, wherein the system provides a selection means 61 for real-time selection of two differential electrodes from the grid (plus, optionally, for one suitable ground electrode). In contrast to the embodiment shown in FIG. 5, this embodiment does not require a differential amplifier for each electrode triplet. Minimally, one differential amplifier 62 is required which receives two differential electrode signals from the selection means 61, plus a signal provided from a ground electrode. The two differential electrodes are selected according to the signal quality they provide, and regardless of their position in the grid. Factors affecting the signal quality provided by the differential electrodes are
quality of galvanic contact to the skin, with bad contact resulting, among others, in high impedance and thus leading to 50/60 Hz noise,
absence or presence of disturbing factors, like skin hair, cosmetic products applied to skin, skin artifacts, like a thick stratum corneum, or enhanced transpiration,
absence or presence of bioelectrical signals interfering with sleep-related bioelectrical signals, like EMG (electromyograms), or EOG (electrooculograms).
   The embodiment according to FIG. 6 offers higher flexibility than the embodiment in which the electrodes are functionally arranged in fixed groups. Thorough selection of the best combination of differential electrodes may result in a better overall signal quality. Further, the technical requirements of this embodiment are less demanding, because only a few channels have to be recorded. This embodiment thus requires less A/D converters (analog-to-digital converters), and less data storage. Furthermore, the system is more flexible, because in case of a sudden decrease in the signal quality of one electrode, e.g., due to system failure or loss of skin contact, a new electrode can be selected in real time. In this embodiment, at least one ground electrode can either be comprised in the grid, too, or located elsewhere on the body or the user, e.g. in the form of a wristband, headband or body electrode, or disposed in a blanket or in a bed linen.

FIG. 7 gives an overview of the EEG electrode nomenclature under the “10-20 system”, which is an internationally recognized method to describe and apply the location of scalp electrodes in the context of an EEG test or experiment. The letters F, T, C, P and O stand for Frontal, Temporal, Central, Parietal, and Occipital, respectively. Note that there exists no “central lobe”, i.e., the “C” letter is used for identification purposes only. Even numbers (2, 4, 6, 8) refer to electrode positions on the right hemisphere, whereas odd numbers (1, 3, 5, 7) refer to those on the left hemisphere. Because in one embodiment of the present invention, the subject's head rests on the device in the side position (see FIG. 1), the positions of the sensor areas arranged on the device can be correlated to EEG electrodes under the 10-20 system. Some of the measurements shown in the experimental section relate, e.g., to the C4 electrode (also called “EOGLeft”), and to the A1 electrode, which serve as an EEG electrode and a reference electrode, respectively. These measurements are called “EOGLeft-A1” herein.

FIG. 8 shows different embodiments of the device capable of serving as a head or face support means, and the sensor areas in a side view. The sensor areas comprise at least one differential electrode and/or at least one sensor disposed in a flexible pad having a conductive surface. FIG. 8a shows one exemplary embodiment in the form of an essentially planar device 81 which adopts the shape of a mattress. The sensor areas 82 are disposed on one side of the device only. FIG. 8b shows another exemplary embodiment in the form of a pillow, or cushion, 83. The sensor areas 84 are disposed on both sides of the pillow, or cushion. In this embodiment a grounded shield 85 is provided to shield the sensor elements from the two sides from one another in order to prevent crosstalk and/or noise. Not shown in FIG. 8B is that the sensor elements can also be disposed in, or on, a cover for such pillow, or cushion. FIG. 8c shows another exemplary embodiment in the form of a hemisphere 86, with sensor elements 87.

### DETAILED DESCRIPTION OF EMBODIMENTS

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

According to the invention, a sleep stage annotation system is provided, said system having (i) a plurality of sensor elements comprising differential electrodes, (ii) at least one ground electrode, (iii) a transmitting means to transmit signals generated by the differential electrodes and the at least one ground electrode to a data recording unit, wherein (iv) at least the sensor elements comprising the differential electrodes are arranged on a device capable of serving as a head or face support means. Preferably, the ground electrode is integrated in one of the sensor elements.

In a preferred embodiment, at least the sensor elements comprising the differential electrodes are arranged in a grid-like manner on said device capable of serving as a head or face support means.

The sensor elements can be disposed on one side, on two sides, or on all sides of said device capable of serving as a head or face support means. In some cases it may be necessary to shield the sensor elements from two sides of said devices by an electrical shield in order to prevent crosstalk and/or noise.

As used herein, the term “differential electrode” refers to an electrode which is read out by a differential input of a differential amplifier. Usually, the two electrodes are called “signal electrodes”, (e.g.: EEG electrode when EEGs are measured) and “reference electrodes” (REF). However, both electrode types may have an identical design, and can be used interchangeably.

In a preferred embodiment, the system further comprises an amplifying means for (i) at least one differential electrode or (ii) at least one pair of differential electrodes. An amplifying means for at least one differential electrode is preferably a voltage follower, also called a unity gain amplifier or buffer amplifier. Such an amplifier transfers a voltage from a first circuit, has a high output impedance level and thus prevents the second circuit from loading the first circuit unacceptably and interfering with its desired operation. Such an amplifier, which may also be called a local amplifier or a 1^{st} stage amplifier, serves to protect the signal and eliminate noise when transmitting the signal generated by the differential electrode to a data recording unit. Differential electrodes combined with such an amplifying means can also be called “active electrodes.”

The amplifying means for at least one pair of differential electrodes is preferably a differential amplifier. As used herein, the term “differential amplifier” relates to a type of electronic amplifier that multiplies the difference between two inputs by a constant factor. Such differential amplifier is preferably used to detect bioelectrical signals recorded by at least two differential electrodes. In this embodiment, each electrode is directly connected to one input of a differential amplifier (one amplifier per pair of electrodes); a common system reference electrode is connected to the other input of each differential amplifier.

As an alternative to said direct connection, the electrodes can be connected to the differential amplifier indirectly, too. This means that the signals first pass the above identified buffer amplifier and are then (i) fed into the differential amplifier (which makes sense in case the differential amplifier is not located on-site, i.e., in the device capable of serving as a head or face support means) or (ii) recorded on a data storage device, and fed into the differential amplifier later for off-line analysis.

The differential amplifiers amplify the voltage difference between the EEG electrode and the reference (typically 1,000-100,000 times, or 60-100 dB of voltage gain). In analog EEG, the signal is then filtered, and the EEG signal is output to an analog display means (e.g., an Oscilloscope, or a pen writer). Most EEG systems, however, are digital, and the amplified signal is digitized via an A/D converter, after being passed through an anti-aliasing filter. A/D sampling typically occurs at 256-512 Hz in a clinical scalp EEG; sampling rates of up to 20 kHz are used in some research applications.

In another preferred embodiment, at least one sensor element further comprises at least one additional sensor selected from the group consisting of temperature sensor, pressure sensor, light sensor, capacitive sensor, microphone, and/or accelerometer. In this context it is important to understand that the term “sensor element”, as used herein, refers to a device which may comprise one electrode and/or one or more sensors, as described above. Therefore, the term “sensor element” does not mean the same as “sensor” herein. In a preferred embodiment, each differential electrode can be combined with such a sensor in a given sensor element.

A pressure sensor may be used to measure pressure exerted to the electrode. A high pressure may be taken as an indication for a good skin contact of the respective differential electrode. This information can be considered for the selection which electrode signal is going to be evaluated. Said pressure sensor can comprise, e.g., a piezo element.

A temperature sensor can be used to measure the body temperature of the subject, e.g., as a contribution to general health monitoring. In another preferred embodiment, the temperature sensor can be used for contact detection of the respective differential electrode, in like manner as the pressure sensor discussed above.

Light sensors can have different purposes, too. They can for example be used for position detection of the subject resting on the device capable of serving as a head or face support means, or for movement detection of the latter. Such light sensors can preferably be infrared (IR) detectors. As IR light is invisible for the human eye, IR background illumination can be used to provide the proper illumination for said detectors, without disturbing the subject.

A capacitive sensor can be used for active noise cancellation.

Microphones can likewise be used for different purposes. One potential use is snoring detection, because snoring is a condition which may seriously affect quality of sleep.

A switch can preferably be embodied as a pressure sensitive switch. In case the surface of a given sensor area is fully covered by a portion of the head of the subject, a good galvanic contact between the differential electrode and the subject's skin can be assumed. Accordingly, said pressure sensitive switch will be activated, and the signals generated by the respective electrode will be considered for analysis and/or recorded. In case a given sensor area has no contact with the subject's head, the pressure sensitive switch will be deactivated, i.e., the respective differential electrode will not be considered. In case there is only slight, or poor, contact between a given sensor area and the subject's head, it can be provided that the said pressure sensitive switch creates a connection with high impedance. Such signal can then be subject to inspection by an operator prior to analysis. The said switch is preferably a spring-loaded contact switch, or a pressure sensor (e.g., a piezo sensor) connected to a relay circuit or a transistor circuit.

Accelerometers have recently been introduced in many consumer devices, like cell phones, etc. They can be used for ballistic cardiography, a method in which the motions of the body caused by the heart beating are recorded by means of an accelerometer (so called ballistocardiogram, or BCG). Further, accelerometers can be used for the measurement of respiration.

In yet another preferred embodiment, at least one differential electrode and/or at least one sensor according to the above description is disposed in a flexible pad having a conductive surface. Said conductive surface preferably comprises a metallic material, e.g., metallic wires provided in the form of a mesh, a woven or a fleece. Such metallic material is, preferably, selected, from the group consisting of silver, silver chloride, gold, platinum, tungsten, or alloys thereof. Alternatively, said conductive surface may comprise an intrinsically conducting polymer (ICP). Said pad can be supported with a foam or other flexible material in order to ensure a good contact between the electrode and the skin of the subject.

In another preferred embodiment, said transmitting means are wireless transmitting means. Such wireless transmitting means can for example be accomplished as a radio-frequency transmission, e.g., under the Bluetooth standard or the WiFi standard, or as an infrared light transmission, e.g., under the IrDa standard or as commonly implemented into television remote controls and similar devices. Other wireless transmission standards can however be used as well.

Further, it is preferred that at least one ground electrode is also arranged on said device capable of serving as a head or face support device. Alternatively, or additionally, to such embodiment, at least one ground electrode can be arranged elsewhere, e.g., in the form of a wristband, headband or body electrode, or arranged on a bed linen on which the subject rests, or a blanket under which the subject rests.

As used herein, the term “device capable of serving as a head or face support means” relates to either an essentially planar device, like a mattress, or to a three dimensional device. Preferably, said device adopts the shape, or form, of a pillow, a hemisphere or a cushion, or a cover for such pillow, hemisphere, or cushion. In such embodiment, the device can gently force the subject to adopt a predetermined position which ensures a good galvanic contact between the skin and the electrodes. Preferably, such pillow or cushion is anatomically shaped to achieve said effect. Preferably, said pillow or cushion, or said cover for such pillow or cushion, is washable. In such embodiment, the active and passive sensor and electrode components are provided in a water proof manner.

In another preferred embodiment, the electrodes are functionally arranged in fixed groups comprising at least two differential electrodes and one ground electrode each. In this embodiment, the functional correlation of at least two differential electrodes and one ground electrode is fixed, i.e., the signals from the respective differential electrodes and one ground electrode are amplified and the resulting signal is then recorded on one channel of a given data storage device. This requires a fixed wiring scheme of the respective electrodes and amplifiers. Said functional correlation may coincide with a fixed spatial arrangement, in which the respective sensors elements comprising the electrodes of each group are arranged, e.g., in vertical columns or horizontal rows. However, in another preferred embodiment, the distribution of the respective sensor elements of each group may be random. Preferably, said groups of electrodes are triplets of two differential electrodes and one ground electrode. In this embodiment, differential amplification can take place on-site, i.e., in the device capable of serving as a head or face support means. In such embodiment, a differential amplifier is integrated in said planar device for each group of electrodes, e.g., for each triplet. Further, in this embodiment, differential amplification takes place in real-time, preferably. Alternatively, the differential amplification can take place off-site, e.g., in the data recording unit. In this case, it is preferably provided that the signals generated by the differential electrodes are fed into voltage follower (unity gain) buffer amplifiers to eliminate noise when transmitting the signals to the data recording unit. After recording, the data sets can be analyzed, and the recording which yields the best signal quality (S/N ratio, appearance of sleep-related signal patterns) can be selected for further analysis. This embodiment requires that all signals generated by the differential amplifiers (e.g., all signals generated by the different groups of electrodes) recorded. Signal analysis and selection of the best electrode combination may then take place off-line. In most cases, a multichannel data logging/recording device is required, which in turn has relatively high data storage demands, plus the requirement of a multiplexer or a plurality of A/D converters. However, this embodiment ensures that the raw data generated by all electrodes can be stored, and reanalyzed at any time. Further, this embodiment provides a relatively simple wiring scheme, and provides redundancy in case some wiring breaks down.

In yet another preferred embodiment, the system provides means for real-time selection of at least two differential electrodes from a plurality of differential electrodes. In this approach, at least two differential electrodes are selected according to the signal quality they provide, and regardless of their position in the device capable of serving as a head or face support means.

Factors affecting the signal quality provided by the differential electrodes are
quality of the galvanic contact to the skin, poor contact results in, among others things, high impedance and thus leads to 50/60 Hz noise,
absence or presence of disturbing factors, like skin hair, cosmetic products applied to the skin, skin artifacts, like a thick stratum corneum, or enhanced transpiration,
absence or presence of bioelectrical signals interfering with sleep-related bioelectrical signals, like EMG (electromyograms), or EOG (electrooculograms).
   In order to better predict the signal quality provided by each differential electrode, signals from temperature sensors and/or pressure sensors combined with the differential electrode can be evaluated, too. In such embodiment, a high pressure exerted to a pressure sensor may be taken as an indication for a good skin contact of the respective differential electrode. Likewise, a given temperature may be taken as an indication for a good skin contact of the respective differential electrode. In another embodiment, the signal quality of each differential electrode, or of random combinations of the differential amplification signal provided by at least two electrodes, can be checked by visual control, or by use of a respective automatic algorithm, in order to select the best combination of electrodes.

In another embodiment, the signal quality of each differential electrode, or of random combinations of the differential amplification signal provided by at least two electrodes, can be checked by means of a respective algorithm, in order to select the best combination of electrodes. This embodiment offers higher flexibility than the embodiment in which the electrodes are functionally arranged in fixed groups comprising at least two differential electrodes and one ground electrode each. Thorough selection of the best combination of differential electrodes may result in a better overall signal quality. Further, the technical requirements of this embodiment are less demanding, because only a few channels have to be recorded. This embodiment thus requires less A/D converters, and less data storage. Furthermore, the system is more flexible, because in case of a sudden decrease in the signal quality of one electrode, e.g., due to system failure or loss of skin contact, a new electrode can be selected in real time. A similar approach is applicable for the selection of the best suited ground electrode. Factors affecting the signal quality provided by the ground electrode are
quality of the galvanic contact to the skin,
distance to 50/60 Hz noise producing devices.
   The at least one ground electrode can be located in said grid, too, and/or elsewhere, e.g. in the form of a wristband, headband or body electrode, or disposed in a blanket or in a bed linen.

In another preferred embodiment, the system further comprises at least one switching or control means for at least one periphery device selected from the group consisting of room heating, air conditioning, room lighting, heating blanket or heating pillow, massage device, alarm clock, alarm device and/or audio device. Such embodiment has particular benefits for a consumer device. According to the actual sleep status, different periphery devices can be switched on or off, or can be controlled, in order to improve the subject's comfort, or to affect his sleep quality. As regards to an alarm clock, the system can control the latter in such a way that it is made sure that the subject is woken up in the light sleep phase as close to the desired wake up time as possible, in order to avoid respective irritations. As regards an alarm device, such device can be used to transmit an alarm signal to a third person in case of an emergency, e.g. to an emergency service, or to relatives of the subject wearing the device.

In yet another preferred embodiment, the system further comprises at least one sleep stage analysis device or sleep coaching device. A sleep stage analysis device, as described herein, is a device which analyses and classifies the sleep of a subject on the basis of biophysical data, e.g., EEG data and/or RHA data (=respiration, heart & actigraphy data). One preferred way of classification is to allocate the different phases of sleep to at least one of REM sleep, or stage 1-4 sleep according to the nomenclature set forth previously. A sleep coaching device, as described herein, is a device which is capable of performing at least one of the following options:
Visualizing personalized sleep graphs;
Visualizing score values with respect to sleep quality;
Visualizing differences between optimal and actual sleep;
Providing information with respect to factors that negatively affect sleep.

In order to meet these objects, the system may comprise at least one item selected from the group consisting of:
Graphical user interface;
Touchscreen;
Audio in- and/or output;
Web-based analytical platform.

The invention further provides a method for sleep stage annotation, in which a method according to any of the aforementioned claims is used. Further, the invention provides the use of a system or a method according to the invention:
for consumer-based sleep annotation, sleep coaching and/or sleep support;
for clinical or pre-clinical patient monitoring;
in post-clinical patient monitoring;
in intensive patent care, and/or;
in coma monitoring.

The system according to the invention is highly beneficial for the said uses, or indications, as it provides a self-sustained device which can be operated by a trained person without need of a general practitioner. Therefore, the device increases the safety of patients which need sleep stage annotation, for example because they have been relocated to their home after a clinical phase, or because they are in a coma.

### EXPERIMENT DESCRIPTION

Six healthy volunteers participated in the study discussed below. They were informed about the objectives of the study and signed a consent form. In a screening phase, selection of participants was based on absence of subjective sleep complaints and regular sleep/wake patterns. Screening was based on two questionnaires: the Sleep Disorders Questionnaire (SDQ) [2] and the Pittsburgh Sleep Quality Index (PSQI) [3]. All selected participants scored within the normal range of the PSQI. Moreover, none of the participants scored higher than the cut-off scores on the subscales for narcolepsy, apnea, restless legs, and psychiatry of the SDQ [2]. Participants entered the sleep laboratory at 21.00 and were prepared for polysomnography. Lights were turned off at around 23.00 h. The waking up signal was given at around 7 h. Sleep recordings and analysis of polysomnographic sleep recordings were obtained during all sleep episodes with a digital recorder (Vitaport-3, TEMEC Instruments B.V., Kerkrade, The Netherlands), and included EEG recordings (F3/A2, F4/A1, C3/A2, C4/A1, O1/A2, O2/A1) obtained with the Sleep BraiNet system (Jordan NeuroScience, San Bernardino, Calif.), electrooculogram (EOG), electrocardiogram (ECG) and chin electromyogram (EMG). Respiratory effort was measured with chest and abdominal belts. The signals were recorded digitally with a sampling frequency of 256 Hz. An assessor from the Siesta group (Salisbury, USA) scored sleep stages in 30 s epochs according to standard criteria [4].

### Methods

Feature Extraction

In the following two subsections we describe data preprocessing and feature extraction for both the RHA and the EEG approaches. 1) RHA features: The raw respiration signal is first low pass filtered (cut-off 0.5 Hz) and then analyzed for individual breaths. Based on a localized min/max filter, local minima and maxima are detected. When found in the right order, they characterize a single breath. Based upon the distribution of identified breath amplitudes in a signal, too small or too large breaths (outliers) are removed. After this preprocessing the RSP signal is characterized by a sequence of breaths. In a similar manner, the ECG signal is low pass filtered (cut-off 5 Hz) and de-trended and individual heart beats are detected using pattern matching. Again, outlier removal is applied and the resulting signal is a sequence of inter beat intervals (IBIs), which has been transformed into (instantaneous) heart rate (in bpm) by taking its reciprocal and multiplying by 60. The actigraphy signal has been low passed and further normalized on a unit interval. In general, sleep is scored in non-overlapping 30-second long intervals (epochs). Thus, features on respiration, heart and actigraphy signals are calculated on a per-epoch basis.

Features in the EEG Approach

The raw signal used for feature extraction in the EEG approach was recorded by electrodes placed at the following three standardized locations: (1) the upper left eye (“EOG L”), (2) behind the left ear and (3) a ground electrode at the neck of the participant. Given this setup for signal extraction we simply had to subtract the signal recorded at the A1 channel from the signal of the EOGL channel. Furthermore, to estimate the power spectral density of each epoch, Welch's method [5] was applied.

FIG. 2 shows results of the Welch's method where the color represents the power at a certain frequency (top plot). To facilitate the interpretation of the relationship between the Welch's power plot(features) and the reference scoring (labels), the bottom plot in the figure shows corresponding hypnogram and the middle plot shows a power plot but specifically for low frequencies which correspond to deeper sleep (“slow wave sleep”, SWS). It is important to notice that the peaks of power in the SWS plot correspond to n3 sleep stages of the hypnogram. For the machine learning part of the EEG approach input/output pairs were constructed in the following manner: for each long epoch, a power spectrum vector was computed which was associated with a sleep stage label. This resulted in about 800 input-output pairs per subject (corresponding to 7 hours of sleep). A two-dimensional visualization of the feature vectors corresponding to each sleep stage can be obtained using the technique t-Distributed Stochastic Neighbor Embedding technique (“t-SNE”) reported in [6]. FIG. 3 depicts such visualization. Every dot represents a power spectrum. The gray values of the dots show how it's labeling by a sleep scorer and spatial grouping of the dots reflects similarities in their feature vectors. In an ideal world, FIG. 3 should show five well separated groups of dots (one per sleep stage), where each group contains dots of a single color. That would mean that extracted feature vectors contain components which uniquely represent certain sleep stages and these components are very different for every particular sleep stage. However in reality that is never the case, partly due to noise present in the signal used for feature extraction, partly due to imperfections in the procedure of feature extraction and certainly by errors made at the stage where ground truth has been determined (sleep stage labeling in this case). Such artifacts can be seen for example in the lower right corner of the figure where a couple of the blue dots (deep sleep) are located in the middle of the cloud of yellow (awake) dots. We consider this particular visualization (or dimension reduction) almost ideal, because clusters of dots representing sleep stages still can be well separated.

RSLVQ Algorithm

Robust Soft Learning Vector Quantization (RSLVQ) is one of many LVQ variants, originally developed by Kohonen [7]. This family of machine learning algorithms has been applied to classification problems in many fields [8] and is characterized by its transparency and computational efficacy. LVQ is a method of prototype-based, multi-class classification, representing each class by one or more prototypes. A prototype is defined as a point in the N-dimensional feature space with an accompanying class label, and trained by sequential handling of training data. Each time a training sample is presented; the closest prototypes with correct and incorrect labels are pulled towards or pushed away from the training sample, respectively. When training progresses, the prototypes will better and better represent the classes. When applied to unseen data, classification is performed by returning the label to the closest prototype. Usually, though not restricted the, Euclidean distance is used as a distance metric. In a recent study [9], the performance of several LVQ variants in a controlled environment was analyzed. The (relative) robustness and convergent properties (i.e., insensitivity to overtraining) motivated our choice for RSLVQ, as proposed in [10]. In this “‘soft” version of LVQ the magnitude of displacement of prototypes in each training step is relative to their distance from the training sample. This method makes an assumption on the distribution of data samples around the prototypes, which we chose to be Gaussian with equal variances (for each prototype). The total distribution of data from a single class therefore is assumed to be a mixture of Gaussian distributions.

Performance Measurement

The results of both experiments were presented in the same format in order to allow more detailed comparisons. Table 2 shows an example of an agreement matrix used for presenting an output of a classifier.

TABLE 2

| Overall | Wake | Light | Deep | Rem | Sum | Sensitivity |
|---|---|---|---|---|---|---|
| Wake | 1206 | 42 | 8 | 42 | 1298 | 92.91% |
| Light | 320 | 1931 | 456 | 430 | 3137 | 61.56% |
| Deep | 21 | 102 | 735 | 15 | 874 | 84.10% |
| Rem | 26 | 118 | 16 | 796 | 956 | 83.26% |
| Sum | 1574 | 2193 | 1215 | 1283 | 6265 | |
| PPV | 76.62% | 88.05% | 60.49% | 62.04% | | |
| Agreement | | | | | | 74.51% |
| Cohen's Kappa | | | | | | 0.64317 |

Table 2 contains the overall results of the classification of the sleep stages obtained from the second data set employing the limited EEG features. Essentially, this agreement matrix contains three widely known (in classification tasks assessments) comparison entities: (1) confusion matrix, (2) percentage of agreement and (3) Cohen's Kappa agreement coefficient. The confusion matrix can be used for detailed assessment of a classifier's performance in terms of which classes are often mistaken for what other classes. Furthermore, they allow calculation of a baseline performance based on just class priors. For this, one takes the 5th row of numbers (the sum of actual label occurrences) and divides the highest number by the total sum, in the case of Table 2, it is 1989/6292=31.61%.

Since we were mostly interested in overall performance assessment, in section IV for each cycle of the cross validation scheme we only present its outcome with two values: (1) the percentage of agreement and (2) Cohen's Kappa coefficient.

Cross Validation Scheme:

In order to determine the generalization ability of the classifiers, we employed leave-one-person-out cross validation. In this procedure n (with n equal to the number of participants) rounds of training and validation are performed, where, in each round, all samples from a single participant are used for validation and the samples of the other n−1 participants are used for training. When finished, all samples have been used for validation exactly once, and the resulting classification performance resembles well the situation in which a product has been pre-trained on a gathered data set and put in use by an unseen user (consumer). This method of validation is the most strict, but also the most fair in the comparison with human raters (compared to e.g. k-fold cross validation), who also do not have participant specific information beforehand.

### Results and Discussion

This section presents the results obtained under two sleep monitoring approaches, namely EEG and RHA. The first subsection reports the EEG results while the second subsection reports the results obtained under the RHA approach. Both subsections contain tables presenting percentages of agreement and Cohen's Kappa coefficients per cross validation run, as well as overall agreement matrixes allowing for detailed assessment of the classifier's performance, and therefore assessment of the quality of extracted features given the classification task. Table 3 shows Cohen's Kappa and percentage of agreement figures per run of the cross-validation scheme. The last column contains average values.

TABLE 3

| | Subjects (Eindhoven data set) | | | | | | |
|---|---|---|---|---|---|---|---|
| | 1 | 2 | 3 | 4 | 5 | 6 | mean |
| Agreement % | 76.20 | 71.43 | 71.46 | 64.71 | 80.91 | 82.07 | 74.46 |
| Cohen's Kappa | 0.66 | 0.58 | 0.62 | 0.50 | 0.69 | 0.74 | 0.63 |

Table 4 shows the overall agreement matrix that contains confusion matrix, (in bold), percentage of agreement, Cohen's Kappa coefficient, positive predictive values (PPV) and sensitivity of the classifier per class.

TABLE 4

| Overall | Wake | Light | Deep | Rem | Sum | Sensitivity |
|---|---|---|---|---|---|---|
| Wake | 1206 | 42 | 8 | 42 | 1298 | 92.91% |
| Light | 320 | 1931 | 456 | 430 | 3137 | 61.56% |
| Deep | 22 | 102 | 735 | 15 | 874 | 84.10% |
| Rem | 26 | 118 | 16 | 796 | 956 | 83.26% |
| Sum | 1574 | 2193 | 1215 | 1283 | 6265 | |
| PPV | 76.62% | 88.05% | 60.49% | 62.04% | | |
| Agreement | | | | | | 74.51% |
| Cohen's Kappa | | | | | | 0.64317 |

Table 4 shows that the overall performance is firmly above random guessing, which is 1989/6292=31.61%. Furthermore, it can be seen for the largest number of confusions is for actual wake epochs being (falsely) recognized as light sleep. Actually, the classifier is falsely biased towards light sleep, as it classifies half of the total number of epochs as light sleep (i.e, 3173/6292=50.43%), resulting in a low sensitivity (52.66%) for that class.

In addition to numerical representation of the classification, FIG. 4 shows both input data (processed EEG spectrum) (FIG. 4B) and hypnograms both target (top) and estimated (bottom) (FIG. 4C).

The top plot of the figure shows the power spectrum of a recording of the signal generated by differential electrodes C4 and A1 (see FIG. 6), which served as an input for an additional experiment we conducted. The essence of the experiment was in substituting the full EEG signal with the C4-A1 signal. Given the fact that the C4 electrode is mounted close to the brain and subsequently has a stronger signal, our assumption was to observe gain in classification performance. However, this experiment proved to have an opposite effect. Despite better signal to noise ratio we registered a significant drop in performance of the classifier, which allowed us to speculate that EEG channel is better suited for sleep stages estimation.

Table 5 shows the overall performance matrix for the recording of the C4-A1 channel. From this Table 5, it is apparent when compared to Table 3 that Cohen's Kappa statistics lowered by 0.0662 and percentage of agreement by 6.55%.

TABLE 5

| Overall | Wake | Light | Deep | Rem | Sum | Sensitivity |
|---|---|---|---|---|---|---|
| Wake | 1199 | 18 | 15 | 66 | 1298 | 92.37% |
| Light | 649 | 1263 | 799 | 426 | 3137 | 40.26% |
| Deep | 44 | 97 | 729 | 4 | 874 | 83.41% |
| Rem | 36 | 10 | 241 | 669 | 956 | 69.98% |
| Sum | 1928 | 1388 | 1784 | 1165 | 6265 | |
| PPV | 62.19% | 90.99% | 40.86% | 57.42% | | |
| Agreement | | | | | | 61.61% |
| Cohen's Kappa | | | | | | 0.49303 |

B. RHA,—respiration, heart and actigraphy signals for hypnogram estimation Table 6 shows Cohen's Kappa and percentage of agreement figures per run of the cross-validation scheme. The last column contains the average values.

TABLE 6

| | Subjects (Boston data set) | | | | | | |
|---|---|---|---|---|---|---|---|
| | 1 | 2 | 3 | 4 | 5 | 6 | mean |
| Agreement % | 48.37 | 33.62 | 27.87 | 32.33 | 43.74 | 13.55 | 33.25 |
| Cohen's Kappa | 0.26 | 0.09 | 0.06 | 0.09 | 0.25 | 0.32 | 0.18 |

Table 7 shows the overall agreement matrix that contains: confusion matrix, (in bold), percentage of agreement, Cohen's Kappa coefficient, positive predictive values (PPV) and sensitivity of the classifier per class.

TABLE 7

| Overall | Wake | Light | Deep | Rem | Sum | Sensitivity |
|---|---|---|---|---|---|---|
| Wake | 247 | 10 | 8 | 73 | 338 | 73.08% |
| Light | 596 | 761 | 723 | 1020 | 3100 | 24.55% |
| Deep | 118 | 158 | 370 | 254 | 900 | 41.11% |
| Rem | 221 | 241 | 53 | 368 | 883 | 41.68% |
| Sum | 1182 | 1170 | 1154 | 1715 | 5221 | |
| PPV | 20.90% | 65.04% | 32.06% | 21.46% | | |
| Agreement | | | | | | 33.44% |
| Cohen's Kappa | | | | | | 0.12265 |

Table 7 shows the agreement figures earlier presented along with the agreement figures achieved by RHA and EEG approaches. From these figures, it is apparent that the EEG approach is superior compared to the RHA approach in both percentages of agreement and Cohen's Kappa coefficient numbers. Figures of the RHA approach show a very low performance of the classifiers when based on respiration, heart and actigraphy features. It can be seen that the overall performance is very close to random guessing, which is 1715=5221=32:85%. Again, the classifier is falsely biased towards light sleep, as it classifies most of the total number of epochs as light sleep (i.e, 3100=5221=59:38%), resulting in a very low sensitivity (24:55%) for class V.

TABLE 8

| | Cohen's Kappa | Agreement (%) |
|---|---|---|
| Human expert (PSG) vs. Human | 0.80 | 86.00 |
| expert (PSG) | | |
| Automated technique (PSG) vs. | 0.69 | 85.48 |
| Automated technique (PSG) | | |
| Automated technique (PSG) vs. | 0.65 | 77.78 |
| MyZeo (EEG head-band) | | |
| Automated technique (PSG) vs. pEEG | 0.64 | 74.51 |
| Automated technique (PSG) vs. RHA | 0.12 | 33.44 |

### CONCLUSION

Based on the experimental results obtained it is concluded that: (1) There is no significant correspondence at individual level (cross subjects) between polysomnography (“PSG”) based sleep stages estimated by experts and the features extracted in the RHA approach. Therefore, these features generally are not separable in terms of sleep stages, which make it hard to design a well-functioning system for sleep stages estimation based solely on RHA. (2) From the product proposition point of view (due to its sensor arrangements) the acquisition of EEG features is not limited by the following drawbacks: (a) privacy considerations (compared to e.g., camera based solutions) and (b) health concerns (as associated with e.g., radar based solutions). (3) In contrast to RHA, classification results obtained on features extracted in the EEG approach look very promising. Visualization of the feature space (see FIG. 3) shows good separability in terms of sleep stages. In the current study, we employed only “simple” (low capacity, memory less, epoch based) classifiers that show a performance of 67% agreement and 0.52 Cohen's Kappa. Although these performance figures might not seem very impressive, they actually are because of the following reasons: (a) Typical experts versus expert agreement figures in average are 88% for agreement and 0.68 Kappa indicating that the ground truth is ill-defined, therefore the goal (in terms of performance) of automated sleep stage classification should be to match the level of the majority of human raters instead of optimizing for a perfect match with a particular rater; (b) Improvement of performance can be expected when we take into account order and transition probabilities between sleep stages. We suggest to focus future research on: (1) For sleep stages estimation in RHA approach: (a) focus on extraction of useful sleep characteristics (not sleep stages) which are directly derivable from the signals employed in RHA approach, and (b) consult physiological signal processing specialists regarding extraction of features (other than described in RHA) with the hope to obtain features that do contain sleep stage information and allow unambiguous sleep stages identification, and (2) For the EEG approach one should focus efforts in three directions: (a) utilize order and transition probabilities between sleep stages; (b) selection of the most suitable classifier; (c) development of a prototype (such as a pillow-array of dry electrodes combined with a data logger) further determine possible applications areas, and (d) initiate work in the direction of continuous “hypnogram” rather than following the standard way of assessing sleep stages in 30 second epochs.

### REFERENCES

[1] J. Virkkala, “Automatic sleep stage classification using electrooculography,” Ph. D. dissertation, Faculty of Computing and Electrical Engineering, Tampere University of Technology, 2005.
[2] A. Douglass, R. Bornstein, G. Ninomurcia, S. Keenan, L. Miles, and V. Zarcone, “THE SLEEP DISORDERS QUESTIONNAIRE-I—CREATION AND MULTIVARIATE STRUCTURE OF SDQ,” Sleep, vol. 17, no. 2, pp. 160-167, 1994.
[3] D. Buysse, C. Reynolds-III, T. Monk, S. Berman, and D. Kupfer, “Pittsburgh sleep quality index: A new instrument for psychiatric practice and research,” Psychiatric Research, vol. 28, no. 2, pp. 193-213, 1989.
[4] A. Rechtschaffen and A. Kales, “A manual of standardized terminology, techniques and scoring system for sleep stages of human subjects,” U.S. National Institute of Neurological Diseases and Blindness, Neurological Information Network, Bethesda, Md., 1968.
[5] P. Welch, “The use of fast Fourier transform for the estimation of power spectra: A method based on time averaging over short, modified periodograms,” IEEE Transactions on Audio Electroacoustics, vol. AU-15, p. 7073, 1967.
[6] L. van der Maaten and G. Hinton, “Visualizing data using t-sne,” Journal of Machine Learning Research, vol. 9, 2008.
[7] T. Kohonen, “Learning vector quantization,” The Handbook of Brain Theory and Neural Networks, M. Arbib, Ed. Cambridge, Mass.: MIT Press, 1995.
[8] N. N. R. Centre, “Bibliography on the self-organizing maps (som) and learning vector quantization (lvq),” 2002. [Online]. Available: http://liinwww.ira.uka.de/bibliography/Neural/SOM.LVQ.html.
[9] A. W. Witoelar, A. Ghosh, J. J. G. de Vries, B. Hammer, and M. Biehl, “Window-based example selection in learning vector quantization,” Neural Computation, vol. 22, no. 11, pp. 2924-2961, 2010.
[10] S. Seo and K. Obermayer, “Soft learning vector quantization,” Neural Computation, vol. 15, pp. 1589-1604, 2003.

1. A sleep stage annotation system, said system having (i) a plurality of sensor elements comprising differential electrodes, (ii) an at least one ground electrode, (iii) a transmitting means to transmit signals generated by said differential electrodes and said at least one ground electrode to a data recording unit, wherein (iv) said plurality of sensor elements comprising the differential electrodes are arranged on a device capable of serving as a head or face support means.

2. The system according to claim 1, wherein said plurality of sensor elements comprising the differential electrodes are arranged in a grid-like manner on said device capable of serving as a head or face support means.

3. The system according to any of the afore-mentioned claims, in which said system further comprises an amplifying means for (i) an at least one differential electrode or (ii) at least one pair of differential electrodes.

4. The system according to any of the aforementioned claims, in which said system at least one sensor element further comprises at least one additional selected from the group consisting of temperature sensor, pressure sensor, light sensor, capacitive sensor, microphone, switch and/or accelerometer.

5. The system according to any of the aforementioned claims, wherein at least one differential electrode and/or at least one sensor according to claim 4 is disposed in a flexible pad having a conductive surface.

6. The system according to any of the aforementioned claims, wherein said transmitting means are wireless transmitting means.

7. The system according to any of the aforementioned claims, wherein at least one ground electrode is also arranged on said device capable of serving as a head or face support device.

8. The system according to any of the aforementioned claims, wherein said device adopts the shape, or form, of a pillow or a cushion, or a cover for such pillow or cushion.

9. The system according to any of the aforementioned claims, wherein the electrodes are functionally arranged in fixed groups comprising at least two differential electrodes and one ground electrode each.

10. The system according to any of the aforementioned claims, wherein the system provides means for real-time selection of at least two differential electrodes from a plurality of differential electrodes.

11. The system according to any of the aforementioned claims, which system further comprises at least one switching or control means for at least one periphery device selected from the group consisting of room heating, air conditioning, room lighting, heating blanket or heating pillow, massage device, alarm clock, alarm device and/or audio device.

12. The system according to any of the aforementioned claims, which system further comprises at least one sleep stage analysis device or sleep coaching device.

13. A method for sleep stage annotation, in which method a system according to any of the aforementioned claims is used.

14. Use of a system according to any of claims 1-12, or a method according to claim 13,
for consumer-based sleep annotation, sleep coaching and/or sleep support;
for clinical or pre-clinical patient monitoring;
in post-clinical patient monitoring;
in intensive patent care, and/or;
in coma monitoring.
